(19) United States
(12) Patent Application Publication
(10) Pub. No.: US 2025/0256026 A1
(43) Pub. Date: Aug. 14, 2025

(54) Device for Early Detection of Pediatric IV Infiltration

(71) Applicant: THE JOHNS HOPKINS UNIVERSITY, Baltimore, MD (US)

(72) Inventors: Taj El-Khalili, Baltimore, MD (US); Sneha Kamada, Poughkeepsie, NY (US); Rebecca Mosier, Baltimore, MD (US); Robin Yang, Baltimore, MD (US)

(21) Appl. No.: 18/857,693

(22) PCT Filed: Apr. 19, 2023
(86) PCT No.: PCT/US2023/065958

§ 371 Date: Oct. 17, 2024

Related U.S. Application Data
(60) Provisional application No. 63/332,570, filed on Apr. 19, 2022.

(51) Int. Cl. A61M 5/168

(52) U.S. Cl. CPC A61M 5/16831; A61M 2205/18; A61M 2205/3327; A61M 2205/3592; A61M 2205/50; A61M 2205/581; A61M 2205/583; A61M 2205/8206

(57) ABSTRACT

A sensor device, according to the present invention, is designed to detect an intravenous therapy (IV) infiltration. The device includes a pressure and/or force sensor placed in between two pressure sensitive adhesive tapes. The sensor is then applied over the IV site to detect IV infiltration. During an IV infiltration, the pressure sensitive adhesive tape will experience force, stretch and/or deform. Stretching is registered as an increase in resistance, because the individual units are spread apart, and electrical continuity is lowered. When the sensor detects a change in resistance, the sensor triggers an alert to a health care provider to check the patient's IV.

### CROSS REFERENCE TO RELATED APPLICATIONS

This application claims the benefit of U.S. Provisional Patent Application No. 63/332,570 filed on Apr. 19, 2022, which is incorporated by reference, herein, in its entirety.

### FIELD OF INVENTION

The present invention relates generally to the field of medical devices. More particularly, the present invention relates to a device for early detection of pediatric IV infiltration.

### BACKGROUND

Every year in the pediatric intensive care unit (PICU) in the United States, between 10-30% of patients will experience an intravenous therapy (IV) infiltration. IV infiltration occurs when the fluid administered leaks out of its intended intravascular path and into surrounding tissue. Failure to detect this promptly can lead to damaging effects such as necrosis and compartment syndrome. Necrosis and compartment syndrome can increase the length of hospital stay and cost of care. Currently, nurses monitor the IV site every few hours for symptoms of swelling, blanching, and change in temperature. However, these symptoms are already indicative of late-stage infiltrations and immediate diagnosis is often delayed, as nurses are unable to monitor the site frequently enough to catch the onset of these complications prior to life threatening damage.

A device for early detection of pediatric IV infiltration that provides constant monitoring of the IV site and an alert to medical personnel is therefore needed.

### SUMMARY

The foregoing needs are met, to a great extent, by the present invention wherein in one aspect, a device for detecting an intravenous therapy (IV) infiltration event in a subject includes an adhesive patch. The adhesive patch is formed from a pressure sensitive adhesive tape. The device also includes a sensor configured to detect changes in resistance based on length. The sensor is embedded in the adhesive patch, such that the sensor does not make direct contact with the subject.

According to an embodiment of the present invention, the sensor is configured to trigger an alert to a healthcare provider about a potential IV infiltration event. The alert is an audible alarm, and/or a visual alert. The sensor transmits data to a processing device, and wherein the processing device is configured to execute an algorithm for determining whether there has been an IV infiltration event.

### BRIEF DESCRIPTION OF THE DRAWINGS

Further objectives and advantages will become apparent from a consideration of the description, drawings, and examples.

FIG. 1 illustrates a perspective view of a prior art IV setup.

FIG. 2A illustrates a perspective view of an IV setup according to an embodiment of the present invention.

FIG. 2B illustrates a top down view of a sensor device according to an embodiment of the present invention.

FIG. 3 illustrates a perspective view of an IV setup, according to an embodiment of the present invention.

FIG. 4 illustrates a perspective view of an IV setup in a patient room, according to an embodiment of the present invention.

FIGS. 5A and 5B illustrate graphical views of voltage measured by the sensor as an infiltration begins.

FIGS. 6 and 7 illustrate flow diagrams for an algorithm, in accordance with an aspect of the present invention.

### DETAILED DESCRIPTION

The presently disclosed subject matter will now be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention provides a sensor device to detect an intravenous therapy (IV) infiltration. The device includes a pressure and/or force sensor placed in between two pressure sensitive adhesive tapes. The sensor is then applied over the IV site in order to detect IV infiltration. During an IV infiltration, the pressure sensitive adhesive tape will experience force, stretch and/or deform. Stretching is registered as an increase in resistance, because the individual units are spread apart and electrical continuity is lowered. When the sensor detects a change in resistance, the sensor triggers an alert to a health care provider to check the patient's IV.

The sensor is made of a conductive stretch cord that changes resistance based on length. The stretch cord is sliced into small units that register force directed both downward and upward on the pressure sensitive adhesive tape. The individual stretch units are placed on the pressure sensitive adhesive tape in a linear pattern such that at rest they are all touching each other. Deformation will also spread out the individual sensor units but also provide an upward force, also increasing the resistance. Even without significant deformation the individual units are sensitive enough to detect force changes underneath the skin resulting from infiltrated fluid.

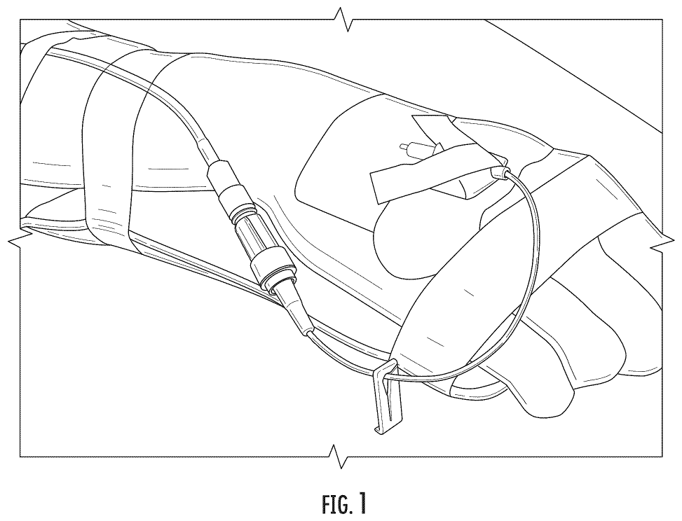

FIG. 1 illustrates a perspective view of a prior art IV setup. In the prior art setup, the IV is placed and held in place with adhesive tape. The adhesive tapes are placed in such a way as to minimize movement of the IV once it is placed in the patient's hand. For instance, the adhesive tapes are placed over the point of insertion, over the hub, and to secure the tubing to the arm of the patient. The patient's hand and wrist can be placed on an armboard to minimize movement. It should be noted that while the prior art setup for the IV is designed to minimize movement of the IV once it is placed, it does not completely prevent an IV infiltration, as fluid commonly leaks out of pediatric veins regardless of catheter disruption. The prior art setup also does not monitor and alert medical personnel if an IV infiltration has occurred.

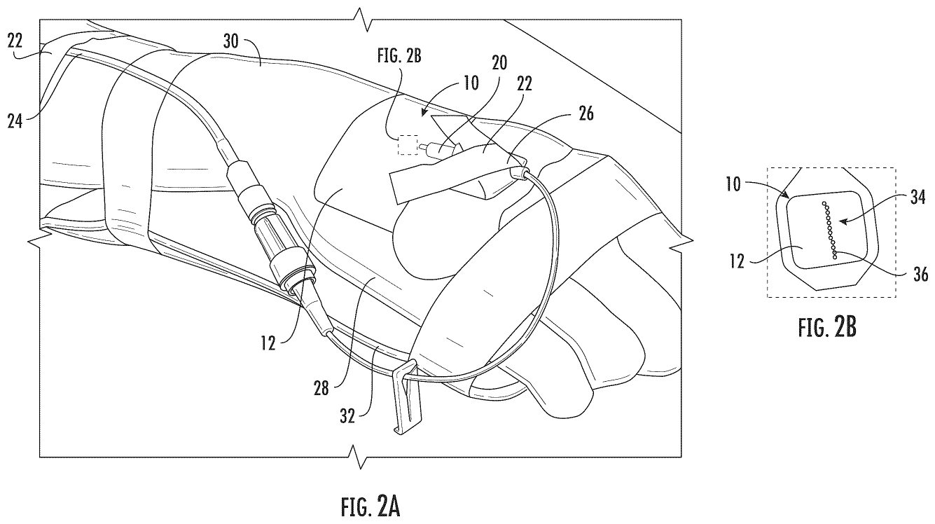

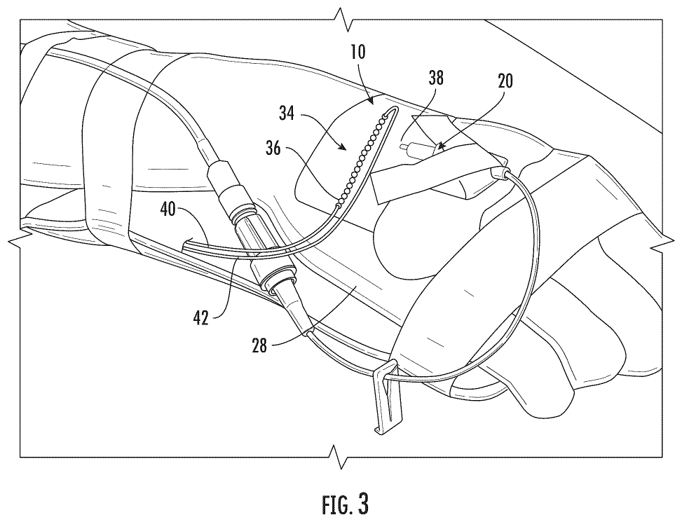

FIG. 2A illustrates a perspective view of an IV setup and IV infiltration detection device, and FIG. 2B illustrates a top-down view of a sensor device according to an embodiment of the present invention. The IV 20 is placed in the same manner, as illustrated in FIG. 1, and can be secured with adhesive tape 22 on the tubing 24 and hub 26. The patient's hand 28 and wrist 30 can be placed on an armboard 32 to further minimize movement. The IV infiltration detection device 10 includes at least one layer of pressure sensitive adhesive tape 12. The pressure sensitive adhesive tape 12 is configured to adhere to the patient's skin, and wherein the pressure sensitive adhesive tape 12 is conformable to the patient's skin. The pressure sensitive adhesive tape 12 can in some embodiments have a biocompatible adhesive printed in a pattern, wherein the adhesive recovery percentage is at least 35%. The adhesive layer can in some embodiments be printed in a lattice pattern and the adhesive layer can further be cross-linked. The adhesive is printed on a film layer that can be formed from a biocompatible plastic, polymer, or other suitable material known to or conceivable to one of skill in the art. One exemplary adhesive tape 12 is Tegaderm™.

As illustrated in FIG. 2B a sensor 34 is embedded in the adhesive tape 12. Therefore, the sensor does not contact the patient's skin. Only the adhesive tape contacts the skin of the patient. Adhesive tapes such as Tegaderm™ stretches with the skin beautifully, so the sensor 34 sits on top of the Tegaderm™ and monitors how the Tegaderm™ deforms. The sensor 34 operates using strain and deformation of the adhesive tape 12 rather than skin strain and deformation. Therefore, the device of the present invention can detect infiltrations that happen anywhere underneath the IV infiltration detection device 10 (the infiltration does not have to occur directly under the sensor). It should be noted that while Tegaderm™ is used as an example, herein, any suitable pressure sensitive adhesive tape known to or conceivable by one of skill in the art can also be used.

Further, as illustrated with respect to FIG. 2B, the sensor 34 can, in some embodiments, be formed of small separable units of resistors 36 that change resistance based on a length of a distance between units 36. The IV infiltration detection device 10 not only monitors strain and deformation of the adhesive tape 12, but also any pressure or compression resulting from edema from the skin pushing upwards onto the patch. In one embodiment, the sensor 34 is formed from a conductive stretch cord that changes resistance based on length. The stretch cord is segmented into small units that register force directed downward and upward on the adhesive tape 12 of the IV infiltration detection device 10. The individual stretch units are placed on the adhesive tape in a linear pattern such that at rest they are all touching each other. During an IV infiltration event, the adhesive tape 12 will experience force, stretch and/or will deform. Stretching is registered as an increase in resistance since the individual units are spread apart and electrical continuity is lowered. Deformation will also spread out the individual units but also provide an upward force, also increasing the resistance. Even with no deformation the individual units are sensitive enough to detect force changes underneath the skin resulting from infiltrated fluid. The device of the present invention is non-invasive and the only contact with the patient is via the adhesive tape. The device can be optimized for pediatric anatomy and is effective in detecting IV infiltration events without a high cost.

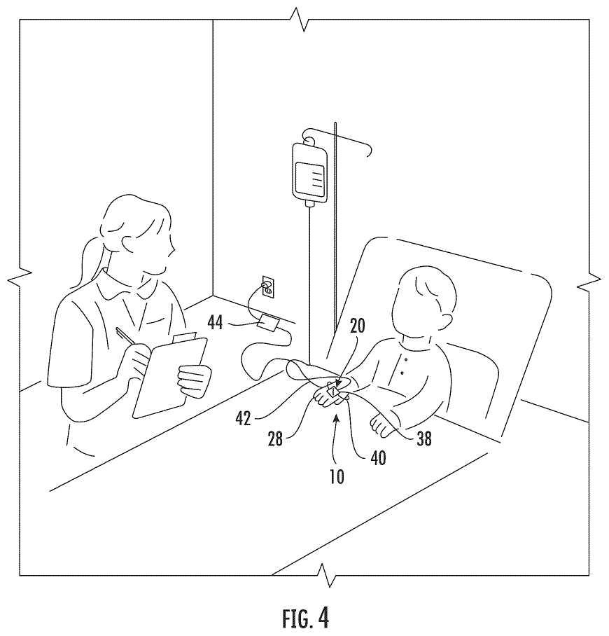

FIG. 3 illustrates a perspective view of an IV setup, according to an embodiment of the present invention. As illustrated in FIG. 3, the IV 20 is placed in the hand 28 of the patient. The IV infiltration detection device 10 of the present invention is placed over the IV insertion point 38. The IV infiltration detection device 10 can include sensor 34. The sensor 34 can take the form of small separable units of resistors 36, as described with respect to FIG. 2B. Wires 40 and 42 are coupled to the IV infiltration detection device 10 in order to receive the signal from the sensor 34 and transmit the signal to a processor, CPU, microprocessor or other device. In other embodiments the IV infiltration detection device 10 is wireless and includes embedded microelectronics for processing and transmitting signal information. In such embodiments, the device can also include a power source, such as a battery.

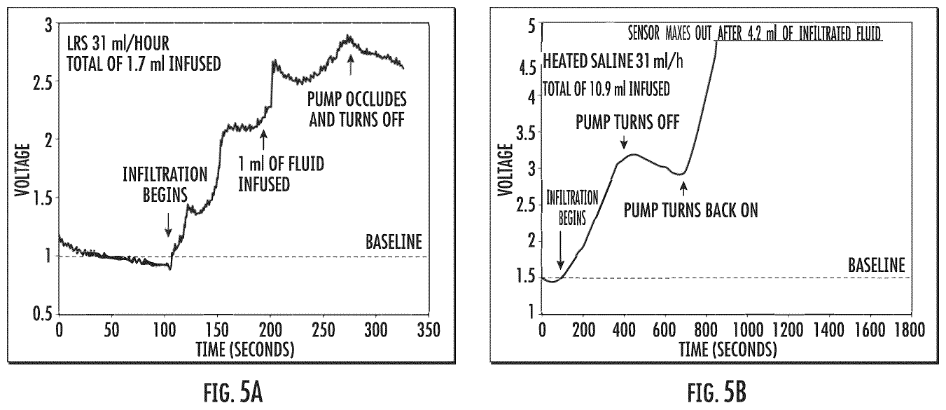

FIG. 4 illustrates a perspective view of an IV setup in a patient room, according to an embodiment of the present invention. As illustrated in FIG. 4, the IV 20 is placed in the hand 28 of the patient. The IV infiltration detection device 10 of the present invention is placed over the IV insertion point 38. Wires 40 and 42 are coupled to the device 10 to receive signal from the sensor 38. The wires are then coupled to a power block and processing component 44. The power block and processing component 44 can include battery power or can be plugged into a wall outlet, as illustrated in FIG. 4. The power block and processing component includes a processor configured to execute an algorithm associated with the present invention. The algorithm takes signal information from the sensor 38 and processes it to determine whether an IV infiltration event is taking place. If the sensor and algorithm detect a possible IV infiltration event an alarm is sounded or an alert is otherwise transmitted to a healthcare provider for further investigation. The alarm or alert can take the form of an audible alert or a visual alert on a computer screen or remote device associated with the care of the patient or with the healthcare provider.

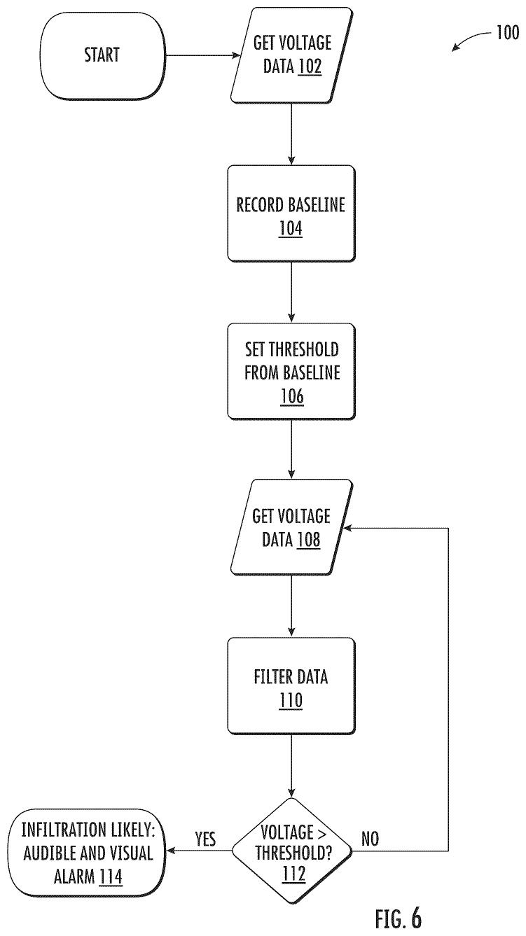

FIGS. 5A and 5B illustrate graphical views of voltage measured by the sensor as an infiltration begins. As illustrated in the graphs of FIGS. 5A and 5B as an IV infiltration event begins the voltage detected by the sensors increases over time. The sensor is sensitive to small changes and increases in fluid underneath the sensor device. Therefore, the device of the present invention is effective in detecting IV infiltration events, and can be used to alert healthcare professionals accordingly.

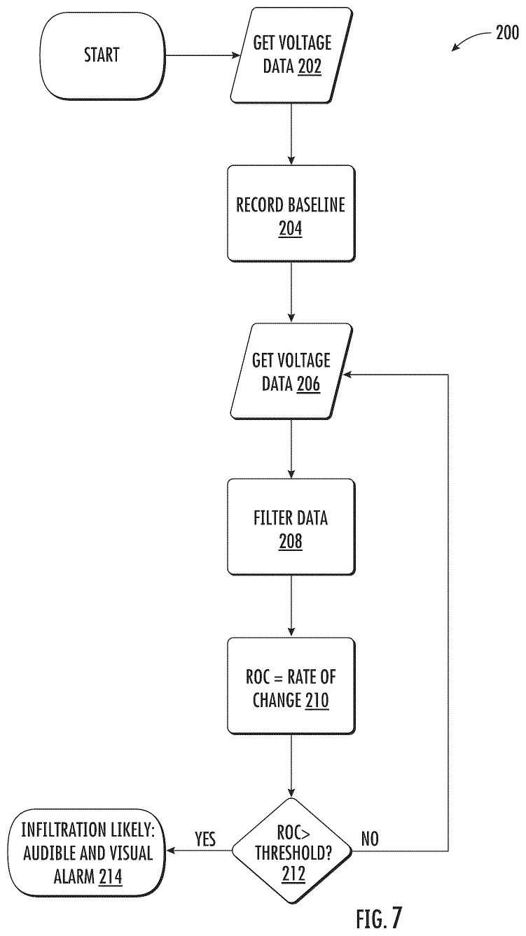

FIGS. 6 and 7 illustrate flow diagrams for an algorithm, in accordance with an aspect of the present invention. The processor of the device executes the algorithm in order to make a determination as to whether or not there has been an IV infiltration event. The algorithm 100 of FIG. 6 receives the voltage data in step 102 and records the baseline in step 104. The processor sets a threshold from the baseline in step 106. The processor continues to receive voltage data at predetermined periods per step 108. The processor filters the data in step 110 and determines whether the voltage is greater than the threshold in step 112. If the voltage is not greater than the threshold, the processor continues to receive the voltage data at predetermined periods per step 108 and the processor repeats steps 110 and 112. If the voltage is greater than the threshold an infiltration event is likely. The device will sound an audible and visual alarm, as described in step 114.

The algorithm 200 of FIG. 7 receives the voltage data in step 202 and records the baseline in step 204. The processor continues to receive voltage data at predetermined periods per step 206. The processor filters the data in step 208. The processor determines a rate of change (ROC) of the voltage in step 210 and then determines whether the ROC is greater than the threshold in step 212. If the voltage is not greater than the threshold, the processor continues to receive the voltage data at predetermined periods per step 206 and the processor repeats steps 208, 210 and 212. If the ROC is greater than the threshold an infiltration event is likely. The device will sound an audible and visual alarm, as described in step 214.

Some prior art devices can match the detection speed of the present invention but are only validated to detect local infiltrations occurring at the sensor's location, to compensate for their sensors taking up a lot of space blocking the IV site for nurses, significantly affecting their workflow. Preserving space and ensuring workflow is especially important in the pediatric context, where the patient is smaller and space is already at a premium. Additionally, most sensors seen are forms of strain gauges that are typically welded to metal to measure strain resulting from permanent deformation and are therefore not well suited for detecting strain of skin or pressure sensitive tape. Some infiltrations do not form a bump until a very late stage of the IV infiltration event (we saw this in our animal studies about 50% of the time), and it is unlikely the strain gauge sensor types can detect these kind of infiltrations before significant damage has occurred.

The device of the present invention can detect infiltrations that form a bump, and infiltrations that do not form a localized bump, both of which can be detected at an early stage. Additionally the bump does not have to be near the sensor, it can be anywhere underneath the dressing. This is because the sensor of the present invention can measure force from deformation at other parts of the dressing. Additionally the present invention can measure force when there is no bump deformation, which occurs when the fluid is infiltrating but spreading out across the tissue instead of staying localized.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It should also be noted that while the present invention is discussed with reference to its important use for detecting pediatric IV infiltration, the present invention can be used in any scenario where an IV is being used. Additionally, while the IV is described with respect to a placement in a hand of a patient, it should be understood that can be placed in any suitable location known to one of skill in the art, and that the device of the present invention can also be placed anywhere an IV is placed. It is therefore to be understood that, within the scope of the claims and their equivalents, the invention may be practiced otherwise than as specifically described.

1. A device for detecting an intravenous therapy (IV) infiltration event in a subject comprising:
an adhesive patch, wherein the adhesive patch is formed from a pressure sensitive adhesive tape; and
a sensor, wherein the sensor is configured to detect changes in resistance based on length, wherein the sensor is embedded in the adhesive patch, such that the sensor does not make direct contact with the subject.

2. The device of claim 1 wherein the sensor is configured to trigger an alert to a healthcare provider about a potential IV infiltration event.

3. The device of claim 2 wherein the alert is an audible alarm.

4. The device of claim 2 wherein the alert is a visual alert.

5. The device of claim 1 wherein the sensor transmits data to a processing device, and wherein the processing device is configured to execute an algorithm for determining whether there has been an IV infiltration event.

6. The device of claim 1 wherein the sensor is configured for wireless transmission of data.

7. The device of claim 6 further comprising a battery.

8. The device of claim 5 wherein the device is coupled to the processing device via a wired connection.

9. The device of claim 5 further comprising a power block.

10. The device of claim 1 where the sensor takes the form of separable units of resistors.

11. The device of claim 1 wherein the sensor is configured to detect changes and increases in fluid under the device.

12. The device of claim 1 wherein the sensor transmits information as voltage data.

13. A device for detecting an intravenous therapy (IV) infiltration event in a subject comprising:
an adhesive patch, wherein the adhesive patch is formed from a pressure sensitive adhesive tape;
a sensor, wherein the sensor is configured to detect changes in resistance based on length, wherein the sensor is embedded in the adhesive patch, such that the sensor does not make direct contact with the subject; and
a processor configured to receive voltage data from the sensor such that changes in the voltage data can be monitored, and wherein voltage data greater than a threshold triggers an alert.

14. The device of claim 13 wherein the alert is an audible alarm.

15. The device of claim 13 wherein the alert is a visual alert.

16. The device of claim 13 wherein the sensor is configured for wireless transmission of data.

17. The device of claim 16 further comprising a battery.

18. The device of claim 13 wherein the device is coupled to the processing device via a wired connection.

19. The device of claim 13 further comprising a power block.

20. The device of claim 13 where the sensor takes the form of separable units of resistors.
